(19) United States
(12) Patent Application Publication
(10) Pub. No.: US 2010/0307522 A1
(43) Pub. Date: Dec. 9, 2010

(54) COSMETIC COMPOSITION COMPRISING AT LEAST ONE AMORPHOUS FILM-FORMING POLYMER AND HAVING A CERTAIN THERMAL PROFILE

(75) Inventors: Karl PAYS, Saint Maurice (FR); Sandrine OLIVIER-MABILAIS, L'Hay Les Roses (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 12/837,239

(22) Filed: Jul. 15, 2010

Related U.S. Application Data
(63) Continuation of application No. 10/821,920, filed on Apr. 12, 2004, now abandoned.

(30) Foreign Application Priority Data
Apr. 11, 2003 (FR) 0304571
Apr. 11, 2003 (FR) 0304593
Apr. 17, 2003 (FR) 0304825
Dec. 19, 2003 (FR) 0351146

(51) Int. Cl. A45D 40/26; A61K 8/92; A61Q 1/10; A46B 11/08

(52) U.S. Cl. 132/200; 424/70.7; 401/1; 132/218

(57) ABSTRACT

The present disclosure relates to a cosmetic composition comprising, in a physiologically acceptable medium, at least one first compound which gives the composition a thermal profile wherein the melting peak has a mid-height width Lf less than or equal to 10° C., and at least one amorphous film-forming polymer capable of forming a water-soluble film, wherein the amorphous film-forming polymer is present in an amount greater than or equal to the amount of the first compound.

The present disclosure also relates to a process for obtaining a film deposited on the keratin fibers which is uniform and/or which has improved curling properties comprising applying the above composition to keratin fibers.

This application claims benefit of U.S. Provisional Application No. 60/536,992, filed Jan. 20, 2004.

The present disclosure relates to a cosmetic composition wherein the thermal profile has a melting peak with a mid-height width (Lf) less than or equal to 20° C., such as less than or equal to 10° C.

The composition according to the present disclosure may be, for example, a cosmetic composition for coating keratin fibers such as human eyelashes, eyebrows and hair, or alternatively false eyelashes. The present disclosure also relates to a process for making up or caring for keratin materials.

The composition as disclosed herein may be a makeup composition, also known as a mascara, a makeup base for keratin fibers, or basecoat, a composition to be applied over a makeup, also known as a topcoat, or a composition for treating keratin fibers. For instance, the composition according to the present disclosure may be a mascara.

“Curling” mascara compositions comprising a mixture of waxes and at least one film-forming polymer are known, such as the compositions described in EP-B-0 928,607.

The use of organogelling agents in mascara compositions that may replace all or some of the waxes in order to possibly obtain improved curling properties of the eyelashes is also known from WO 00/74519.

However, such compositions do not always allow optimum curling of the eyelashes.

Moreover, devices exist to assist with curling the eyelashes, such as “eyelash curlers.” One type of eyelash curler consists, for example, in pinching the eyelashes between the jaws of a clip to give them a curled shape before applying the makeup, but this operation can be difficult to perform.

Some other eyelash curlers come in the form of a heating clip or a heating brush, as described in U.S. Pat. No. 5,853,010 or JP 2000-38314, to shape the eyelash by the action of heat. These heating instruments may be applied to naked eyelashes, but the curling effect obtained can be poor, or applied to eyelashes coated with any mascara composition. In the case where the heating instrument is applied to coated eyelashes, it is common for some of the composition coating the eyelashes to be removed by the action of the heat and/or for the cosmetic properties of this composition to be degraded. For example, the composition film can lose its homogeneity, and the eyelashes can become stuck together, which results in an unattractive makeup result on the keratin fibers.

An aim of the present inventors is to provide a composition for coating keratin fibers that allows improved curling of the eyelashes, such as by the action of heat, and which provides at least one good cosmetic property chosen, for example, from uniform deposition and good curling behavior over time.

This composition may be used for instance, in combination with a heating instrument, such as a heating brush, which may be applied to the eyelashes before, during or after they have been coated with the composition, or contained in a device for applying the composition while hot.

The inventors have discovered that a mascara having at least one of the properties described above may be obtained by using a composition with a particular thermal profile.

For example, one aspect of the present disclosure is a cosmetic composition comprising, in a physiologically acceptable medium:
i) at least one compound, referred to as the “first compound”, which gives the cosmetic composition a thermal profile wherein the melting peak has a mid-height width Lf less than or equal to 10° C., and
ii) at least one amorphous film-forming polymer capable of forming a water-soluble film, wherein the at least one amorphous film-forming polymer is present in an amount greater than or equal to the amount of the first compound.

For purposes of the present disclosure, the term “physiologically acceptable medium” means a non-toxic medium that can be applied to keratin fibers, such as human eyelashes, eyebrows and hair, and which is for instance, compatible with the region of the eyes.

Another aspect of the present disclosure is a process for coating keratin fibers in order to deposit a film which has a uniform appearance and improved curling properties, comprising applying to the keratin fibers a cosmetic composition comprising, in a physiologically acceptable medium:
i) at least one first compound which gives the cosmetic composition a thermal profile wherein the melting peak has mid-height width Lf less than or equal to 10° C., and
ii) at least one amorphous film-forming polymer capable of forming a water-soluble film, wherein the at least one amorphous film-forming polymer is present in an amount greater than or equal to the amount of the first compound.

Still another aspect of the present disclosure is a non-therapeutic cosmetic process for making up or caring for keratin fibers, comprising the application to the keratin fibers of a composition as defined above.

A further aspect of the present disclosure is a non-therapeutic cosmetic process for making up or caring for keratin materials, such as the skin, the lips or keratin fibers, comprising the application to the keratin materials of a cosmetic composition comprising, in a physiologically acceptable medium:
i) at least one first compound which gives the cosmetic composition a thermal profile wherein the melting peak has a mid-height width Lf less than or equal to 20° C.,
ii) at least one amorphous film-forming polymer capable of forming a water-soluble film,
   wherein the cosmetic composition being, prior to, simultaneously with or subsequent to its application, brought to a temperature above or equal to its melting point m.p., and for instance, above or equal to its end melting temperature Tf.

Yet another aspect of the present disclosure is also a non-therapeutic cosmetic process for making up or caring for keratin materials, comprising the application to the keratin materials of a cosmetic composition comprising at least one amorphous film-forming polymer capable of forming a water-soluble film, the composition being, simultaneously with or subsequent to its application, brought to a temperature above or equal to its melting point, and for example, a temperature above or equal to its end melting temperature. The composition may be brought to a temperature above or equal to its melting point simultaneously with or subsequent to its application, for instance, with an application device comprising heating means, such as a heating brush.

### Determination of the Thermal Profile of the Composition

The thermal profile of the composition according to the present disclosure is determined using a differential scanning calorimeter (DSC), for example the calorimeter sold under the name DSC 30 by the company Mettler.

A sample of 5 to 10 mg of product placed in a crucible is subjected to a first temperature increase ranging from −20° C. to 90° C., at a heating rate of 5° C./minute, it is then cooled from 90° C. to −20° C. at a cooling rate of 5° C./minute and is then subjected to a second temperature increase ranging from −20° C. to 90° C. at a heating rate of 5° C./minute. During the second temperature increase, the variation of the difference in power absorbed by the empty crucible and by the crucible containing the sample of product is measured as a function of the temperature.

The thermal profile of the composition according to the present disclosure shows at least one narrow melting peak, which for example, has a mid-height peak width Lf of less than or equal to 20° C., such as less than or equal to 10° C.

In the case where the thermal profile of the composition comprises several melting peaks, the “melting peak of the composition” is considered as the melting peak whose Lf is less than or equal to 20° C., such as less than or equal to 10° C., having the lowest starting melting temperature. The melting point, temperature amplitude (ΔT), starting melting temperature and end melting temperature parameters described below are determined from this peak.

For example, the thermal profile of the composition according to the present disclosure has a melting peak wherein the mid-height peak width Lf ranges from 0.5° C. to 20° C., such as from 0.5 to 10° C., from 1° C. to 10° C., and from 2° C. to 5° C.

The mid-height of the melting peak may be determined on the basis of the half-distance between a straight line connecting two flat portions of the thermal profile on either side of the melting peak, and the top of the peak.

The melting peak may also have a melting point m.p. ranging from 20° C. to 80° C., for instance, ranging from 25° C. to 75° C., and from 35° C. to 60° C. The melting point of the peak that is considered is the temperature value corresponding to the top of the peak of the curve representing the variation of the difference in power absorbed as a function of the temperature.

The melting peak may, for example, have a low temperature amplitude, ΔT=Tf−To, of less than or equal to 30° C., such as ranging from 1° C. to 30° C., from 2° C. to 25° C., and from 3° C. to 20° C., wherein To is the starting melting temperature corresponding to the temperature measured when 5% of the heat of fusion is consumed, and wherein Tf is the end melting temperature, which corresponds to the temperature measured when 95% of the heat of fusion has been consumed.

For example, the starting melting temperature To of the composition may be greater than or equal to 10° C., for example ranging from 10° C. to 50° C., greater than or equal to 15° C., for example ranging from 15° C. to 45° C., and greater than or equal to 20° C., for example ranging from 20° C. to 40° C.

For instance, the end melting temperature of the composition Tf may be less than or equal to 90° C., for example ranging from 35° C. to 90° C., less than or equal to 80° C., for example ranging from 40° C. to 80° C., and less than or equal to 70° C., for example ranging from 40° C. to 70° C., and from 40 to 60° C.

Compositions having such characteristics, for example, a low amplitude ΔT, can have the particular feature, when they are heated to a temperature above their melting point m.p., for instance to a temperature above or equal to their end melting temperature (Tf), of passing from a supple or soft state, i.e., temperature above m.p. to a semi-rigid or semi-crystalline state, i.e., temperature below m.p. in a relatively short space of time.

Thus, it is possible to shape eyelashes coated with such a composition by the action of a source of heat, such as a heating brush with a temperature above or equal to the melting point of the composition, and to do so relatively quickly.

For instance, the thermal profile of the composition according to the present disclosure may have a single melting peak.

Compound Giving the Composition a Thermal Profile Wherein the Melting Peak has a Mid-Height Width Lf Less than or Equal to 20° C.

For example, the at least one compound giving the composition a thermal profile wherein the melting peak has mid-height width Lf less than or equal to 20° C., such as less than or equal to 10° C., i.e., the first compound, may itself have a thermal profile wherein the melting peak has a mid-height width Lf less than or equal to 20° C., such as less than or equal to 10° C.

The at least one first compound may be, for example, chosen from waxes, semi-crystalline polymers and oils thickened with a structuring agent.

The at least one first compound may be present in the composition in an amount ranging from 1% to 60%, such as from 3% to 55%, for instance from 5% to 50%, and from 10% to 40% by weight, relative to the total weight of the composition.

In the present disclosure, a wax is a lipophilic compound, which is solid at room temperature, i.e., 25° C., with a reversible solid/liquid change of state, which has a melting point of greater than or equal to 30° C., and which may be up to 120° C. By bringing the wax to the liquid state, i.e., melting, it is possible to make it miscible with oils and to form a microscopically homogeneous mixture, but on returning the temperature of the mixture to room temperature, recrystallization of the wax in the oils of the mixture may be obtained.

The melting point of the wax may be measured using a differential scanning calorimeter (DSC), for example the calorimeter sold under the name DSC 30 by the company Mettler. A 15 mg sample of product placed in a crucible is subjected to a first temperature rise ranging from 0° C. to 120° C., at a heating rate of 10° C./minute, and is then cooled from 120° C. to 0° C. at a cooling rate of 10° C./minute, and is next subjected to a second temperature rise ranging from 0° C. to 120° C. at a heating rate of 5° C./minute. During the second temperature rise, the variation in the difference in power absorbed by the empty crucible and by the crucible containing the sample of product is measured as a function of the temperature. The melting point of the compound is the value of the temperature corresponding to the top of the peak of the curve representing the variation of the difference in power absorbed as a function of the temperature.

The waxes that may be used in the composition according to the present disclosure may be chosen from waxes that are solid and rigid at room temperature, of animal, plant, mineral and synthetic origin, and mixtures thereof. The waxes may have a melting point ranging from 30° C. to 80° C., such as ranging from 30° C. to 70° C., and from 35° C. to 65° C.

For example, the wax may be chosen from olive wax obtained by hydrogenation of olive oil esterified with stearyl alcohol such as the wax Phytowax Olive 18L57 (melting point m.p.=58.6° C.) sold by the company Sophim, stearyl alcohol (melting point m.p.=60° C.), stearyl stearate (melting point m.p.=57° C.), stearyl benzoate (melting point m.p.=40° C.), bis(trimethylolpropane) tetrastearate (melting point m.p.=46° C.), the wax Licowax KST (polyethoxylated fatty acids of Montan wax) from the company Clariant (melting point m.p.=55° C.), bis(trimethylolpropane) tetrabehenate (melting point m.p.=67.5° C.) and dioctadecyl carbonate wax (melting point m.p.=57° C.), and mixtures thereof.

The at least one first compound may be present in an amount ranging from 1% to 60%, such as, for example, from 3% to 55%, from 5% to 50% and from 10% to 40% by weight, relative to the total weight of the composition.

The composition according to the present disclosure may comprise, for example, at least one semi-crystalline polymer.

Accordingly, one aspect of the present disclosure is a cosmetic composition comprising, in a physiologically acceptable medium

at least one semi-crystalline first polymer, which gives the composition a thermal profile wherein the melting peak has a mid-height width Lf less than or equal to 20° C., and

at least one amorphous film-forming polymer capable of forming a water-soluble film.

Another aspect of the present disclosure is also a process for obtaining a film deposited on the keratin fibers which provides at least one cosmetic property to the fibers chosen from uniform appearance and improved curling properties, comprising applying to the keratin fibers a cosmetic composition comprising, in a physiologically acceptable medium:

at least one semi-crystalline first polymer which gives the cosmetic composition a thermal profile wherein the melting peak has a mid-height width Lf less than or equal to 20° C., and

at least one amorphous film-forming polymer capable of forming a water-soluble film.

For purposes of the present disclosure, the term “semi-crystalline polymer” means polymers comprising at least one crystallizable portion chosen from crystallizable pendent chains and crystallizable blocks in the skeleton, as well as at least one amorphous portion in the skeleton, wherein the semi-crystalline polymer has a first-order reversible temperature of change of phase, for instance of melting, i.e., solid-liquid transition. When the at least one crystallizable portion is in the form of a crystallizable block of the polymer skeleton, the amorphous portion of the polymer is in the form of an amorphous block; the semi-crystalline polymer is, in this case, a block copolymer, for example of the diblock, triblock or multiblock type, comprising at least one crystallizable block and at least one amorphous block. For purposes of the present disclosure, the term “block” generally means at least five identical repeating units. The at least one crystallizable block is then of different chemical nature from the at least one amorphous block.

The at least one semi-crystalline polymer that may be used in the composition as disclosed herein may have a melting point of greater than or equal to 20° C., such as, ranging from 20° C. to 80° C., from 30° C. to 70° C., and from 35° C. to 65° C. This melting point is a first-order temperature of change of state.

The melting point may be measured by any known method, for example, using a differential scanning calorimeter (DSC) as described above.

The at least one semi-crystalline polymer as disclosed herein, may, for example, have a number-average molecular mass of greater than or equal to 1000. For example, the at least one semi-crystalline polymer of the composition as disclosed herein may have a number-average molecular mass M̅n ranging from 2,000 to 800,000, such as from 3,000 to 500,000, and from 4,000 to 150,000, and less than 100,000, for instance from 4,000 to 99,000. The at least one semi-crystalline polymer may have for instance, a number-average molecular mass of greater than 5,600, for example ranging from 5,700 to 99,000.

For the purposes of the present disclosure, the expression “crystallizable chain or block” means a chain or block which, if it were obtained alone, would change from the amorphous state to the crystalline state reversibly, depending on whether the chain or block is above or below the melting point. For the purposes of the present disclosure, a “chain” is a group of atoms, which are pendent or lateral relative to the polymer skeleton. A “block” is a group of atoms belonging to the skeleton. The “pendent crystallizable chain” may, for example, be a chain comprising at least 6 carbon atoms.

For instance, the at least one crystallizable block and/or chain of the at least one semi-crystalline polymer may be present in the polymer in an amount of at least 30% by weight, relative to the total weight of each polymer, for example, at least 40% by weight. The semi-crystalline polymers as disclosed herein comprising crystallizable blocks are block or multiblock polymers. They may be obtained by polymerizing a monomer containing reactive, or ethylenic, double bonds or by polycondensation. When the semi-crystalline polymers as disclosed herein are polymers comprising crystallizable side chains, these side chains may be, for example, in random or statistical form.

For instance, the semi-crystalline polymers that may be used in the composition as disclosed herein may be of synthetic origin. Moreover, they do not comprise a polysaccharide skeleton. In general, the at least one crystallizable portion, i.e., chains or blocks, of the semi-crystalline polymers according to the present disclosure originate from at least one monomer comprising at least one crystallizable block or chain, used for the manufacture of the semi-crystalline polymers.

Among the semi-crystalline polymers that may be used in the composition according to present disclosure, for example, non-limiting mention may be made of:

block copolymers of polyolefins with controlled crystallization, for example, those whose monomers are described in EP-A-0 951,897,

polycondensates, for instance of aliphatic or aromatic polyester type or of aliphatic/aromatic copolyester type,

homopolymers or copolymers comprising at least one crystallizable side chain and homopolymers or copolymers comprising at least one crystallizable block in the skeleton, for instance those described in U.S. Pat. No. 5,156,911,

homopolymers or copolymers comprising at least one crystallizable side chain, for instance comprising at least one fluoro group, as described in WO-A-01/19333,

and mixtures thereof. With respect to the last twotypes of semi-crystalline polymers described, the crystallizable side chains or blocks are hydrophobic.

### Semi-Crystalline Polymers Comprising Crystallizable Side Chains

Among the semi-crystalline polymers comprising crystallizable side chains, non-limiting mention may be made, for example, of those defined in U.S. Pat. No. 5,156,911 and in WO-A-01/19333. The semi-crystalline polymers may be homopolymers or copolymers comprising from 50% to 100% by weight of units resulting from the polymerization of at least one monomers bearing a crystallizable hydrophobic side chain.

These homopolymers or copolymers may be of any nature, provided that they meet the conditions as disclosed above.

Moreover, these homopolymers or copolymers may result from:

the polymerization, such as the free-radical polymerization, of at least one monomer containing at least one reactive or ethylenic double bond with respect to a polymerization, such as a vinyl, (meth)acrylic or allylic group,

the polycondensation of at least one monomer comprising co-reactive groups, for example, carboxylic acid, sulphonic acid, alcohol, amine or isocyanate, such as, for example, polyesters, polyurethanes, polyethers, polyureas or polyamides.

In general, the at least one semi-crystalline polymers may be chosen from, for example, homopolymers and copolymers resulting from the polymerization of at least one monomer comprising at least one crystallizable chain of formula (I):

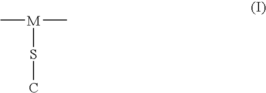

wherein M is chosen from an atom of the polymer skeleton, S is a spacer and C is chosen from crystallizable groups.

The crystallizable chains “—S—C” may be aliphatic or aromatic, and optionally fluorinated or perfluorinated. “S” may be, for example, chosen from (CH₂)ₙ, (CH₂CH₂O)ₙ, and (CH₂O) groups, which may be linear or branched or cyclic, wherein n is an integer ranging from 0 to 22. For example, “S” may be a linear group. For further example, “S” and “C” may be different from each other.

When the crystallizable chains “—S—C” are hydrocarbon-based aliphatic chains, they may comprise hydrocarbon-based alkyl chains comprising at least 11 carbon atoms and not more than 40 carbon atoms, such as no more than 24 carbon atoms. They may be, for instance, aliphatic chains or alkyl chains comprising at least 12 carbon atoms, such as C₁₄-C₂₄ alkyl chains. When they are fluoroalkyl or perfluoroalkyl chains, they may comprise at least six fluorinated carbon atoms, such as at least 11 carbon atoms wherein at least six of the 11 carbon atoms are fluorinated.

As examples of semi-crystalline polymers or copolymers comprising at least one crystallizable chain, non-limiting mention may be made of those resulting from the polymerization of at least one of the following monomers: (meth)acrylates of saturated alkyl with the alkyl group being C₁₄-C₂₄, perfluoroalkyl (meth)acrylates with a C₁₁-C₁₅ perfluoroalkyl group, N-alkyl(meth)acrylamides with the alkyl group being C₁₄ to C₂₄ optionally with a fluorine atom, vinyl esters comprising alkyl or perfluoro(alkyl) chains with the alkyl group being C₁₄ to C₂₄ with at least 6 fluorine atoms per perfluoroalkyl chain, vinyl ethers comprising alkyl or perfluoro(alkyl) chains with the alkyl group being C₁₄ to C₂₄ and at least 6 fluorine atoms per perfluoroalkyl chain, C₁₄ to C₂₄ alpha-olefins such as, for example, octadecene, para-alkylstyrenes with an alkyl group comprising from 12 to 24 carbon atoms, and mixtures thereof.

When the semi-crystalline polymers result from a polycondensation, the hydrocarbon-based and/or fluorinated crystallizable chains as defined above are borne by a monomer that may be a diacid, a diol, a diamine or a diisocyanate.

When the semi-crystalline polymers are copolymers, they may additionally comprise from 0 to 50% of groups Y or Z resulting from the copolymerisation of Y and/or Z, wherein:

Y may be chosen from polar and non-polar monomers, or a mixture of the two.

When Y is a polar monomer, it may be either a monomer bearing polyoxyalkylenated groups such as oxyethylenated and/or oxypropylenated groups; a hydroxyalkyl (meth)acrylate, for instance hydroxyethyl acrylate; (meth)acrylamide; an N-alkyl(meth)acrylamide; an N,N-dialkyl(meth)acrylamide such as, for example, N,N-diisopropylacrylamide or N-vinylpyrrolidone (NVP); N-vinylcaprolactam; a monomer bearing at least one carboxylic acid group, for instance (meth)acrylic acid, crotonic acid, itaconic acid, maleic acid or fumaric acid; or bearing a carboxylic acid anhydride group, for instance maleic anhydride, and mixtures thereof.

When Y is a non-polar monomer, it may be an ester of the linear, branched or cyclic alkyl (meth)acrylate type, a vinyl ester, an alkyl vinyl ether, an alpha-olefin, styrene or styrene substituted with a C₁ to C₁₀ alkyl group, for instance α-methylstyrene, or a macromonomer of the polyorganosiloxane type containing vinyl unsaturation.

For the purposes of the present disclosure, the term “alkyl” means a saturated group, for instance of C₈ to C₂₄, except where noted otherwise, such as of C₁₄ to C₂₄.

Z may be a polar monomer or a mixture of polar monomers. Z has the same definition as that of Y when Y is polar as defined above.

For example, the semi-crystalline polymers comprising a crystallizable side chain may be alkyl (meth)acrylate or alkyl(meth)acrylamide homopolymers with an alkyl group as defined above, for instance of C₁₄-C₂₄, copolymers of these monomers with a hydrophilic monomer preferably of different nature from (meth)acrylic acid, for instance N-vinylpyrrolidone or hydroxyethyl (meth)acrylate, and mixtures thereof.

### Semi-Crystalline Polymers Comprising in the Skeleton at Least One Crystallizable Block

The semi-crystalline polymers comprising in the skeleton at least one crystallizable block may be for example, block copolymers comprising at least two blocks of different chemical nature, one of which is crystallizable. Among such polymers, non-limiting mention may be made of:

the block polymers defined in U.S. Pat. No. 5,156,911;

block copolymers of olefin or of cycloolefin comprising a crystallizable chain, for instance those derived from the block polymerization of:
cyclobutene, cyclohexene, cyclooctene, norbornene (i.e. bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene, or mixtures thereof,
with ethylene, propylene, 1-butene, 3-methyl-1-butene, 1-hexene, 4-methyl-1-pentene, 1-octene, 1-decene or 1-eicosene, or mixtures thereof,

for example, copoly(ethylene/norbornene) blocks and (ethylene/propylene/ethylidene-norbornene) block terpolymers. For further example, the block polymers resulting from the block copolymerization of at least two C₂-C₁₆ α-olefins, such as C₂-C₁₂ α-olefins and C₄-C₁₂ α-olefins, such as those mentioned above and for instance block bipolymers of ethylene and of 1-octene may also be used.

The copolymers may be copolymers comprising at least one crystallizable block, with the copolymer residue being amorphous at room temperature. The copolymers may also contain two crystallizable blocks of different chemical nature. For example, the copolymers may simultaneously comprise at room temperature a crystallizable block and an amorphous block that are both hydrophobic and lipophilic, sequentially distributed; non-limiting mention may be made, for example, of polymers comprising at least one crystallizable block and at least one amorphous block chosen from:

Blocks that are crystallizable by nature may be chosen from polyester, for instance poly(alkylene terephthalate); polyolefin, for instance polyethylenes, or polypropylenes.

Amorphous and lipophilic blocks may be chosen from, for instance, amorphous polyolefins or copoly(olefin)s such as poly(isobutylene), hydrogenated polybutadiene or hydrogenated poly(isoprene).

As examples of such copolymers comprising at least one crystallizable block and at least one separate amorphous block, non-limiting mention may be made of:

poly(ε-caprolactone)-b-poly(butadiene) block copolymers, for instance, used in hydrogenated form, such as those described in the article “Melting behaviour of poly(E-caprolactone)-block-polybutadiene copolymers” from S. Nojima, Macromolecules, 32, 3727-3734 (1999);

the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995);

the poly(ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997); and

the poly(ethylene)-b-poly(ethylethylene) block copolymers cited in the general article “Crystallization in block copolymers” by I. W. Hamley, Advances in Polymer Science, Vol. 148, 113-137 (1999).

The semi-crystalline polymers in the composition of the according to the present disclosure may optionally be partially crosslinked, provided that the degree of crosslinking does not interfere with their dissolution or dispersion, when heated above their melting point, in the liquid fatty phase optionally present in the composition. The crosslinking may be a chemical crosslinking, by reaction with a multifunctional monomer during the polymerization. It may also be a physical crosslinking which may, in this case, be due either to the establishment of bonds of hydrogen or dipolar type between groups borne by the polymer, such as, for example, the dipolar interactions between carboxylate ionomers, these interactions being of small amount and borne by the polymer skeleton; or to a phase separation between the crystallizable blocks and the amorphous blocks borne by the polymer.

For example, the semi-crystalline polymers in the composition as disclosed herein may be non-crosslinked.

According to one aspect of the present disclosure, the at least one polymer may be chosen from copolymers resulting from the polymerization of at least one monomer comprising a crystallizable chain chosen from saturated C₁₄ to C₂₄ alkyl (meth)acrylates, C₁₁ to C₁₅ perfluoroalkyl (meth)acrylates, C₁₄ to C₂₄ N-alkyl(meth)acrylamides optionally with a fluorine atom, vinyl esters comprising C₁₄ to C₂₄ alkyl or perfluoroalkyl chains, vinyl ethers comprising C₁₄ to C₂₄ alkyl or perfluoroalkyl chains, C₁₄ to C₂₄ alpha-olefins, para-alkylstyrenes with an alkyl group comprising from 12 to 24 carbon atoms, with at least one optionally fluorinated C₁ to C₁₀ monocarboxylic acid ester or amide which may be of formula (II):

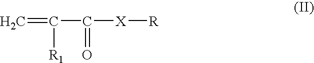

wherein R₁ is chosen from hydrogen atoms and CH₃ groups, R is chosen from optionally fluorinated C₁-C₁₀ alkyl groups, and X is chosen from oxygen atoms, and NH and NR₂ groups wherein R₂ is chosen from optionally fluorinated C₁-C₁₀ alkyl groups.

According to another aspect of the present disclosure, the at least one polymer is derived from a monomer comprising a crystallizable chain, chosen from saturated C₁₄ to C₂₂ alkyl (meth)acrylates.

As examples of the semi-crystalline polymers that may be used in the composition according to the present disclosure, non-limiting mention may be made of the products Intelimer® from the company Landec, described in the brochure “Intelimer® polymers.” These polymers are in solid form at room temperature, 25° C., They also bear crystallizable side chains and have the formula (I) above.

The semi-crystalline polymers may be, for example, those described in Examples 3, 4, 5, 7, 9 and 13 of U.S. Pat. No. 5,156,911 comprising a —COOH group, resulting from the copolymerization of acrylic acid and of C₅ to C₁₆ alkyl (meth)acrylate. For further example, the polymers may be those of the copolymerization:

of acrylic acid, of hexadecyl acrylate and of isodecyl acrylate in a 1/16/3 weight ratio,

of acrylic acid and of pentadecyl acrylate in a 1/19 weight ratio,

of acrylic acid, of hexadecyl acrylate and of ethyl acrylate in a 2.5/76.5/20 weight ratio,

of acrylic acid, of hexadecyl acrylate and of methyl acrylate in a 5/85/10 weight ratio,

of acrylic acid and of octadecyl methacrylate in a 2.5/97.5 weight ratio,

of hexadecyl acrylate, of polyethylene glycol methacrylate monomethyl ether containing 8 ethylene glycol units, and of acrylic acid in an 8.5/1/0.5 weight ratio.

It is also possible to use the “Structure O” from National Starch, a methacrylic acid/octadecyl acrylate (5/85) copolymer with a melting point of 44° C., described in U.S. Pat. No. 5,736,125, and also semi-crystalline polymers with crystallizable pendent chains comprising fluoro groups, described in Examples 1, 4, 6, 7 and 8 of WO-A-01/19333.

It is also possible to use alkyl (meth)acrylate copolymers comprising polydimethylsiloxane grafts, such as the stearyl acrylate copolymer comprising polydimethylsiloxane grafts (melting point of about 30° C.) or the behenyl acrylate copolymer comprising polydimethylsiloxane grafts (melting point of about 49° C.), which are sold by the company Shin-Etsu under the respective names KP-561 and KP 562 (CTFA name: acrylates/dimethicone).

The semi-crystalline polymers obtained by copolymerization of stearyl acrylate and of acrylic acid or of NVP, as described in U.S. Pat. No. 5,519,063 or EP-A-550,745, may also be used.

The semi-crystalline polymers obtained by copolymerization of behenyl acrylate and of acrylic acid or of NVP, as described in U.S. Pat. No. 5,519,063 and EP-A-550,745, may also be used.

### Amorphous Film-Forming Polymer

The at least one amorphous film-forming polymer of the composition according to the present disclosure is capable of forming a water-soluble film.

For purposes of the present disclosure, the expression “amorphous film-forming polymer capable of forming a film” means a polymer capable of forming at room temperature, 25° C., by itself or in the presence of an auxiliary film-forming agent, a continuous film that adheres to a support, for instance to keratin materials.

For purposes of the present disclosure, the expression “polymer or composition capable of forming a water-soluble film” means a polymer or a composition capable of forming a film that is soluble in water or in a mixture of water and of linear or branched lower monoalcohols comprising from 2 to 5 carbon atoms, for instance ethanol, isopropanol and n-propanol, without a change in pH, and at a polymer or composition active material content that is at least greater than 1%, such as greater than or equal to 2% and greater than or equal to 5% by weight, relative to the total weight of the composition at room temperature (25° C.) and atmospheric pressure.

The at least one amorphous film-forming polymer that may be used in the present disclosure may be a copolymer or a homopolymer. The amorphous polymer may be a thermoplastic polymer.

For the purposes of the present disclosure, the term “amorphous polymer or compound” means a polymer that does not have a first-order solid/liquid change of state. This means that the polymer is not characterized by a melting temperature. This characteristic may be confirmed by any known method for example, by DSC.

The amorphous polymer may have, for example, a glass transition temperature (Tg) of greater than or equal to 25° C., for instance greater than or equal to 30° C., greater than or equal to 38° C., greater than or equal to 40° C., greater than or equal to 50° C., and which may be up to 120° C. The glass transition temperature may be measured by DSC.

The at least one amorphous polymer may have, for example, a weight-average molecular mass of less than 200,000, for example ranging from 10,000 to 50,000.

The at least one amorphous polymer may be chosen from polyesters that may be obtained, in a known manner, by polycondensation of at least one dicarboxylic acid with at least one polyol, such as diols.

The at least one dicarboxylic acid may be aliphatic, alicyclic or aromatic. Non-limiting examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid and 2,6-naphthalenedicarboxylic acid. These dicarboxylic acid monomers may be used alone or as a combination of at least two dicarboxylic acid monomers. Among these monomers, non-limiting mention may be made of phthalic acid, isophthalic acid and terephthalic acid.

The at least one diol may be chosen from aliphatic, alicyclic and aromatic diols. The at least one diol may also be, for example, chosen from: ethylene glycol, diethylene glycol, triethylene glycol, 1,3-propanediol, cyclohexanedimethanol and 4-butanediol. Other polyols that may be used include glycerol, pentaerythritol, sorbitol and trimethylolpropane.

The polyesteramides may be obtained in a manner analogous to that of the polyesters, by polycondensation of diacids with diamines or amino alcohols. Diamines that may be used include ethylenediamine, hexamethylenediamine and meta- or para-phenylenediamine. An amino alcohol that may be used is monoethanolamine.

The polyester may also comprise at least one monomer bearing at least one group —SO₃M, wherein M is chosen from hydrogen atoms, ammonium ions NH₄⁺ and metal ions such as, for example, an Na⁺, Li⁺, K⁺, Mg²⁺, Ca²⁺, Cu²⁺, Fe²⁺ or Fe³⁺ ion. For example, a difunctional aromatic monomer comprising a —SO₃M group may also be used.

The aromatic nucleus of the difunctional aromatic monomer also bearing a group —SO₃M as described above may be chosen, for example, from benzene, naphthalene, anthracene, biphenyl, oxybiphenyl, sulphonylbiphenyl and methylenebiphenyl nuclei. As examples of difunctional aromatic monomers also bearing a group —SO₃M, non-limiting mention may be made of sulphoisophthalic acid, sulphoterephthalic acid, sulphophthalic acid, and 4-sulphonaphthalene-2,7-dicarboxylic acid.

Further examples of the copolymers that may used include those based on isophthalate/sulphoisophthalate, for instance, copolymers obtained by condensation of diethylene glycol, cyclohexanedimethanol, isophthalic acid and sulphoisophthalic acid. Such polymers are sold, for example, under the brand name Eastman AQ® by the company Eastman Chemical Products, such as AQ 29S with a Tg equal to 29° C., AQ 55S with a Tg equal to 55° C., AQ 38S with a Tg equal to 38° C., and AQ 48 Ultra with a Tg equal to 48° C., and mixtures thereof.

The solubility of the polymer in aqueous medium, i.e., water or a mixture of water and aqueous-alcoholic solvents, may be adjusted by partial or total neutralization of the pendent sulphonic chains.

As amorphous film-forming polymers capable of forming a water-soluble film, non-limiting mention may also be made of:

proteins, for instance proteins of plant origin such as wheat proteins and soybean proteins; proteins of animal origin such as keratins, for example keratin hydrolysates and sulphonic keratins;

anionic, cationic, amphoteric or nonionic chitin or chitosan polymers;

polymers of cellulose such as hydroxyethylcellulose, hydroxypropylcellulose, methylcellulose, ethylhydroxyethylcellulose and carboxymethylcellulose, and quaternized cellulose derivatives;

acrylic polymers or copolymers, such as polyacrylates or polymethacrylates, for example the sodium polymethacrylate manufactured or sold by the company Vanderbilt under the commercial reference Darvan 7;

vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol; and

polymers of natural origin, which are optionally modified, such as gum arabics, guar gum, xanthan derivatives, karaya gum; alginates and carrageenans; glycosaminoglycans, hyaluronic acid and derivatives thereof; shellac resin, sandarac gum, dammar resins, elemi gums and copal resins; deoxyribonucleic acid; mucopolysaccharides such as hyaluronic acid and chondroitin sulphates,

and mixtures thereof.

The at least one amorphous film-forming polymer may be present in an amount ranging from 0.1% to 40% by weight, such as from 5% to 30% by weight, and from 10% to 20% by weight, relative to the total weight of the composition.

For example, the at least one amorphous film-forming polymer may be present in an amount at least equal to the amount of the at least one semi-crystalline polymer. For instance, the at least one semi-crystalline polymer and the at least one amorphous film-forming polymer may be present in a weight ratio of amorphous film-forming polymer to semi-crystalline polymer ranging from 0.3 to 3, such as from 0.6 to 2, and from 0.9 to 1.5.

The composition may also comprise an oil or an oil thickened with a structuring agent.

In the case where the composition comprises semi-crystalline polymers as described above, these polymers may act as structuring agents. The structuring agent may also be chosen from the lipophilic gelling agents conventionally used in cosmetics.

For purposes of the present disclosure, the term “oil” means a fatty substance that is liquid at room temperature, 25° C., and atmospheric pressure, 760 mmHg, i.e. 105 Pa.

The oil may be chosen from any physiologically acceptable and for instance, cosmetically acceptable oils, such as mineral, animal, plant or synthetic oils; for example volatile or non-volatile hydrocarbon-based and/or silicone and/or fluoro oils, and mixtures thereof. More specifically, for purposes of the present disclosure, the term “hydrocarbon-based oil” means an oil mainly comprising carbon and hydrogen atoms and optionally at least one functional group chosen from hydroxyl, ester, ether and carboxylic functional groups. Generally, the oil may have a viscosity ranging from 0.5 to 100,000 cps, such as from 50 to 50,000 cps, and from 100 to 300,000 cps.

As examples of oils that may be used in the present disclosure, non-limiting mention may be made of:

hydrocarbon-based oils of animal origin, such as perhydrosqualene;

hydrocarbon-based plant oils such as liquid triglycerides of fatty acids of from 4 to 24 carbon atoms, for instance heptanoic or octanoic acid triglyceride, or alternatively sunflower oil, maize oil, soybean oil, marrow oil, grapeseed oil, sesame seed oil, hazelnut oil, apricot oil, macadamia oil, castor oil, avocado oil, caprylic/capric acid triglycerides, for instance those sold by the company Stearineries Dubois or those sold under the names Miglyol 810, 812 and 818 by the company Dynamit Nobel, jojoba oil and shea butter;

linear or branched hydrocarbons of mineral or synthetic origin, such as liquid paraffin and derivatives thereof, petroleum jelly, polydecenes, polybutenes and hydrogenated polyisobutene such as parleam;

synthetic esters and ethers, for example of fatty acids, for instance the oils of formula R₁COOR₂ wherein R₁ is chosen from higher fatty acid residues comprising from 1 to 40 carbon atoms and R₂ is chosen from hydrocarbon-based chains comprising from 1 to 40 carbon atoms wherein R₁+R₂≧10, for instance purcellin oil, isononyl isononanoate, isopropyl myristate, 2-ethylhexyl palmitate, 2-octyldodecyl stearate, 2-octyldodecyl erucate, isostearyl isostearate or tridecyl trimellitate; hydroxylated esters, for instance isostearyl lactate, octyl hydroxystearate, octyldodecyl hydroxystearate, diisostearyl malate, triisocetyl citrate and fatty alkyl heptanoates, octanoates or decanoates; polyol esters, for instance propylene glycol dioctanoate, neopentyl glycol diheptanoate or diethylene glycol diisononanoate; and pentaerythritol esters, for instance pentaerythrityl tetraisostearate;

fatty alcohols comprising from 12 to 26 carbon atoms, for instance octyldodecanol, 2-butyloctanol, 2-hexyldecanol, 2-undecylpentadecanol or oleyl alcohol;

fluoro oils, optionally partially hydrocarbon-based and/or silicone-based;

silicone oils, for instance volatile or non-volatile, linear or cyclic polydimethylsiloxanes (PDMSs); polydimethylsiloxanes comprising alkyl, alkoxy or phenyl groups, which may be pendent or at the end of a silicone chain, these groups comprising from 2 to 24 carbon atoms; phenyl silicones, for instance phenyl trimethicones, phenyl dimethicones, phenyl trimethylsiloxy diphenyl siloxanes, diphenyl dimethicones, diphenyl methyldiphenyl trisiloxanes or 2-phenyl ethyl trimethyl siloxysilicates,

and mixtures thereof.

As lipophilic gelling agents conventionally used in cosmetics, non-limiting examples that may be mentioned include mineral lipophilic gelling agents such as clays or silicas, polymeric organic lipophilic gelling agents such as partially or totally crosslinked elastomeric organopolysiloxanes, block copolymers, of the polystyrene/copoly(ethylene-propylene) type and polyamides, and mixtures thereof.

The composition according to the present disclosure may be in the form of an aqueous or anhydrous continuous phase, or in the form of a water-in-oil or oil-in-water emulsion or a water-in-oil or oil-in-water dispersion.

The total fatty phase of the composition, formed from the semi-crystalline polymer and/or the oil thickened with a structuring agent and an additional fatty substance, may be present in the composition according to the present disclosure in an amount ranging from 0.1% to 60% by weight, for instance ranging from 0.5% to 50% by weight, and from 1% to 40% by weight, relative to the total weight of the composition.

The composition according to the present disclosure may also comprise an aqueous phase, which may consist essentially of water. It may also comprise a mixture of water and of water-miscible solvent, for instance lower monoalcohols comprising from 1 to 5 carbon atoms, such as ethanol or isopropanol, glycols comprising from 2 to 8 carbon atoms, such as propylene glycol, ethylene glycol, 1,3-butyleneglycol or dipropylene glycol, C₃-C₄ ketones and C₂-Cₐ aldehydes. The aqueous phase, i.e., water and optionally the water-miscible organic solvent, may be present in an amount ranging from 5% to 95% by weight, relative to the total weight of the composition.

The aqueous phase of the composition may be thickened with a thickener. Among the aqueous-phase thickeners that may be used according to the present disclosure, non-limiting mention may be made of cellulose-based thickeners, clays, polysaccharides, acrylic polymers and associative polymers, and mixtures thereof.

Hydrophilic thickeners that may be mentioned for example, include the AMPS/acrylamide copolymers of Sepigel or Simulgel type sold by the company SEPPIC.

In the composition as disclosed herein, the aqueous-phase thickener may be present in an amount ranging from 0.1% to 15% by weight, such as from 1% to 10%, for instance, from 1% to 5% by weight, relative to the total weight of the composition.

The composition may further comprise nonionic, anionic, cationic or amphoteric surfactants or alternatively emulsifying surfactants. Reference may be made to the document “Encyclopedia of Chemical Technology, Kirk-Othmer”, Volume 22, pp. 333-432, 3rd edition, 1979, Wiley, for the definition of the properties and emulsifying functions of surfactants, for instance pp. 347-377 of the said reference, for the anionic, amphoteric and nonionic surfactants.

Among the surfactants that may be used in the composition as disclosed herein, non-limiting mention may be made of, for example:
nonionic surfactants chosen from fatty acids, fatty alcohols, polyethoxylated or polyglycerolated fatty alcohols such as polyethoxylated stearyl or cetylstearyl alcohol, fatty acid esters of sucrose, alkyl glucose esters, for instance polyoxyethylenated fatty esters of a C₁-C₆ alkyl glucose, and mixtures thereof, fatty acid esters of glycerol, such as glyceryl mono- and distearate (Tegin M from the company Goldschmidt), esters of fatty acids, such as a C₈-C₂₄ and for instance a C₁₆-C₂₂ acid, and of polyethylene glycol, which may comprise from 1 to 150 ethylene glycol units, for instance oxyethylenated (30 EO) glyceryl monostearate (Tagat S from the company Goldschmidt),
anionic surfactants chosen from C₁₆-C₃₀ fatty acids neutralized with amines, ammonia or alkaline salts, and mixtures thereof.

The composition according to the present disclosure may also further comprise at least one dyestuff, for instance pulverulent dyestuffs, liposoluble dyes and water-soluble dyes. This at least one dyestuff may be present in an amount ranging from 0.1% to 20% by weight, such as ranging from 1% to 15% by weight, relative to the total weight of the composition.

The pulverulent dyestuffs may be chosen from pigments and nacres.

The pigments may be chosen from white or colored, mineral and/or organic, and coated or uncoated pigments. Among the mineral pigments which may be used, non-limiting mention may be made of titanium dioxide, optionally surface-treated, zirconium oxide, zinc oxide or cerium oxide, as well as iron oxide, chromium oxide, manganese violet, ultramarine blue, chromium hydrate and ferric blue. Among the organic pigments that may be used, non-limiting mention may be made of carbon black, pigments of D & C type, and lakes based on cochineal carmine or on barium, strontium, calcium or aluminium.

The nacres may be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the abovementioned type, and nacreous pigments based on bismuth oxychloride.

The liposoluble dyes may be chosen from, for example, Sudan Red, D&C Red 17, D&C Green 6, β-carotene, soybean oil, Sudan Brown, D&C Yellow 11, D&C Violet 2, D&C Orange 5, quinoline yellow and annatto. The water-soluble dyes are, for example, beetroot juice or methylene blue.

The composition according to the present disclosure may also comprise at least one additional film-forming polymer that is different from the amorphous film-forming polymer capable of forming a water-soluble film and from the semi-crystalline polymer.

For purposes of the present disclosure, the term “film-forming” means a polymer capable of forming, by itself or in the presence of an auxiliary film-forming agent, a continuous film that adheres to a support, such as to keratin materials.

The film-forming polymer may be dispersed in the form of solid particles in an aqueous phase of the composition, or may be dissolved or dispersed in the form of solid particles in a liquid fatty phase. The composition may comprise a blend of these polymers. When the film-forming polymer is in the form of solid particles, these particles may have a mean particle size ranging from 5 nm to 600 nm, such as from 20 nm to 300 nm.

The at least one additional film-forming polymer may be present in the composition as disclosed herein in a solids content amount ranging from 0.1% to 60% by weight, such as from 0.5% to 40% by weight, for instance, from 1% to 30% by weight, relative to the total weight of the composition.

Among the additional film-forming polymers that may be used in the composition of the present disclosure, non-limiting mention may be made of synthetic polymers, of radical-mediated type or of polycondensate type, and polymers of natural origin, and mixtures thereof.

For purposes of the present disclosure, the expression “radical-mediated film-forming polymer” means a polymer obtained by polymerization of monomers containing unsaturation, such as ethylenic unsaturation, wherein each monomer is capable of homopolymerizing, unlike polycondensates. The film-forming polymers of radical-mediated type may be, for example, vinyl polymers or copolymers, such as acrylic polymers. The vinyl film-forming polymers can result from the polymerization of monomers containing ethylenic unsaturation and comprising at least one acidic group and/or esters of these acidic monomers and/or amides of these acidic monomers.

Monomers bearing an acidic group which may be used include α,β-ethylenic unsaturated carboxylic acids such as acrylic acid, methacrylic acid, crotonic acid, maleic acid or itaconic acid. For example, (meth)acrylic acid and crotonic acid may be used. In one aspect of the present disclosure, (meth)acrylic acid is used.

The esters of acidic monomers may be chosen from, for example, (meth)acrylic acid esters also known as (meth)acrylates, such as (meth)acrylates of an alkyl, for instance of a C₁-C₃₀ and such as C₁-C₂₀ alkyl, (meth)acrylates of an aryl, for instance of a C₆-C₁₀ aryl, and (meth)acrylates of a hydroxyalkyl, such as of a C₂-C₆ hydroxyalkyl.

Among the alkyl (meth)acrylates that may be used, non-limitin mention may be made of methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates that may be used, non-limiting mention may be made of hydroxyethyl acrylate, 2-hydroxypropyl acrylate, hydroxyethyl methacrylate and 2-hydroxypropyl methacrylate.

Among the aryl (meth)acrylates that may be used, non-limiting mention may be made of benzyl acrylate and phenyl acrylate.

For example, the (meth)acrylic acid esters may be alkyl (meth)acrylates.

The alkyl group of the esters may be either fluorinated or perfluorinated, i.e. some or all of the hydrogen atoms of the alkyl group may be substituted with fluorine atoms.

Among examples of amides of the acid monomers that may be used, non-limiting mention may be made of (meth)acrylamides, such as N-alkyl(meth)acrylamides, for instance of a C₂-C₁₂ alkyl. Among the N-alkyl(meth)acrylamides that may be used, non-limiting mention may be made of N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For instance, these monomers may be polymerized with acid monomers and/or esters thereof and/or amides thereof, such as those mentioned above.

Non-limiting examples of vinyl esters that may be mentioned include vinyl acetate, vinyl neodecanoate, vinyl pivalate, vinyl benzoate and vinyl t-butylbenzoate.

Non-limiting examples of styrene monomers that may be mentioned include styrene and α-methylstyrene.

It is possible to use any monomer known to those skilled in the art that falls within the categories of acrylic and vinyl monomers, including monomers modified with a silicone chain.

Among the film-forming polycondensates that may be used, non-limiting mention may be made of polyurethanes, polyesters, polyesteramides, polyamides, epoxyester resins and polyureas.

The polyurethanes may be chosen from anionic, cationic, nonionic and amphoteric polyurethanes, polyurethane-acrylics, polyurethane-polyvinylpyrrolidones, polyester-polyurethanes, polyether-polyurethanes, polyureas and polyurea/polyurethanes, and mixtures thereof.

The polymers of natural origin, optionally modified, may be chosen from shellac resin, sandarac gum, dammar resins, elemi gums, copal resins and cellulose polymers, and mixtures thereof.

According to one aspect of the present disclosure, the at least one additional film-forming polymer may be present in the form of particles dispersed in an aqueous phase, which is generally known as a latex or pseudolatex. The techniques for preparing these dispersions are known to those skilled in the art.

Aqueous dispersions of film-forming polymers that may be used include the acrylic dispersions sold under the names Neocryl XK-90®, Neocryl A-10700, Neocryl A-1090®, Neocryl BT-62®, Neocryl A-1079® and Neocryl A-523® by the company Avecia-Neoresins, Dow Latex 432® by the company Dow Chemical, Daitosol 5000 AD® by the company Daito Kasey Kogyo; Syntran 5760, Syntran 5190 and Syntran 5170 sold by the company Interpolymer or the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer and Aquamere H-1511® by the company Hydromer

Aqueous dispersions of film-forming polymer that may also be used include the polymer dispersions resulting from the free-radical polymerization of at least one free-radical monomer inside and/or partially at the surface of pre-existing particles of at least one polymer chosen from polyurethanes, polyureas, polyesters, polyesteramides and/or alkyd polymers. These polymers are generally referred to as hybrid polymers.

According to another aspect of the composition according to the present disclosure, the additional film-forming polymer may be present in a liquid fatty phase comprising organic oils or solvents. For the purposes of the present disclosure, the expression “liquid fatty phase” means a fatty phase which is liquid at room temperature, 25° C., and atmospheric pressure, 760 mm Hg, i.e. 10⁵ Pa, composed of at least one fatty substance that is liquid at room temperature, also known as oils, which are generally mutually compatible.

The liquid fatty phase may comprise, for example, a volatile oil, optionally mixed with a non-volatile oil, the oils possibly being chosen from those mentioned above.

According to still another aspect of the composition according to the present disclosure, the film-forming polymer may be present in the form of surface-stabilized particles dispersed in the liquid fatty phase.

The dispersion of surface-stabilized polymer particles may be manufactured as described in EP-A-749,747.

The polymer particles may be surface-stabilized by means of a stabilizer, which may be a block polymer, a grafted polymer and/or a random polymer, alone or as a mixture.

Dispersions of film-forming polymer in the liquid fatty phase, in the presence of stabilizers, are described for example, in EP-A-749,746, EP-A-923,928 and EP-A-930,060, the content of which is incorporated into the present disclosure by reference.

The size of the polymer particles in dispersion either in the aqueous phase or in the liquid fatty phase may range from 5 nm to 600 nm, such as from 20 nm to 300 nm.

According to still yet another aspect of the composition according to the present disclosure, the film-forming polymer may be dissolved in the liquid fatty phase, in which case the film-forming polymer is said to be a liposoluble polymer.

As examples of liposoluble polymers which may be used, non-limiting mention may be made of copolymers of vinyl ester the vinyl group being directly linked to the oxygen atom of the ester group and the vinyl ester comprising a saturated, linear or branched hydrocarbon-based radical of 1 to 19 carbon atoms, linked to the carbonyl of the ester group, and of at least one other monomer which may be a vinyl ester other than the vinyl ester already present, an α-olefin comprising from 8 to 28 carbon atoms, an alkyl vinyl ether in which the alkyl group comprises from 2 to 18 carbon atoms, or an allylic or methallylic ester comprising a saturated, linear or branched hydrocarbon-based radical of 1 to 19 carbon atoms, linked to the carbonyl of the ester group.

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Non-limiting examples of these copolymers which may be mentioned include: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetaallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Non-limiting examples of liposoluble film-forming polymers that may also be mentioned include liposoluble homopolymers, such as those resulting from the homopolymerization of vinyl esters comprising from 9 to 22 carbon atoms or of alkyl acrylates or methacrylates, and alkyl radicals comprising from 10 to 20 carbon atoms.

Such liposoluble homopolymers may be chosen from polyvinyl stearate; polyvinyl stearate crosslinked with the aid of divinylbenzene, of diallyl ether or of diallyl phthalate; polystearyl (meth)acrylate, polyvinyl laurate and polylauryl (meth)acrylate, it being possible for these poly(meth)acrylates to be crosslinked with the aid of ethylene glycol dimethacrylate or tetraethylene glycol dimethacrylate.

The liposoluble homopolymers and copolymers defined above are known and are described for example, in patent application FR-A-2,262,303; they may have a weight-average molecular weight ranging from 2,000 to 500,000, such as from 4,000 to 200,000.

As liposoluble film-forming polymers which may be used as disclosed herein, non-limiting mention may also be made of polyalkylenes and for instance, copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) and for example, copolymers of vinylpyrrolidone and of C₂ to C₄₀, such as C₃ to C₂₀ alkene. As examples of VP copolymers which may be used according to the present disclosure, non-limiting mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

The composition according to the present disclosure may comprise an auxiliary film-forming agent that promotes the formation of a film with the at least one film-forming polymer. Such a film-forming agent may be chosen from any compound known to those skilled in the art as being capable of satisfying the desired function, such as plasticizers and coalescers.

The composition of the present disclosure may also comprise at least one additive usually used in cosmetics, such as fillers, antioxidants, preserving agents, fragrances, neutralizers, plasticizers, cosmetic active agents, for instance emollients, moisturizing agents, vitamins and sunscreens, and mixtures thereof. The at least one additive may be present in the composition in an amount ranging from 0.01% to 10% by weight, relative to the total weight of the composition.

The fillers may be chosen from those that are known to a person skilled in the art and commonly used in cosmetic compositions. The fillers may be chosen from mineral, organic, lamellar and spherical fillers. Non-limiting mention may be made of talc, mica, silica, kaolin, polyamide powder for instance Nylon® (Orgasol from Atochem), poly-β-alanine powder and polyethylene powder, tetrafluoroethylene polymer powders for instance Teflon®, lauroyllysine, starch, boron nitride, expanded hollow polymer microspheres such as those made of polyvinylidene chloride/acrylonitrile, for instance Expancel® (Nobel Industrie), acrylic powders such as Polytrap® (Dow Corning), polymethyl methacrylate particles and silicone resin microbeads (for example Tospearls® from Toshiba), precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids comprising from 8 to 22 carbon atoms such as from 12 to 18 carbon atoms, for example zinc, magnesium or lithium stearate, zinc laurate or magnesium myristate.

The fillers may be present in an amount ranging from 0.1% to 25%, such as from 1% to 20% by weight, relative to the total weight of the composition.

Needless to say, a person skilled in the art will take care to select the optional additional additives and/or the amount thereof such that the advantageous properties of the composition according to the present disclosure are not adversely affected by the envisaged addition, for example, such that the thermal profile of the composition remains as described above.

The composition according to the present disclosure may be manufactured by the known processes generally used in cosmetics.

The composition according to the present disclosure may be intended to be heated.

The heating of the composition, for instance, in order to curl the eyelashes, may be performed after applying the product, for example using devices as described U.S. Pat. No. 5,853,010.

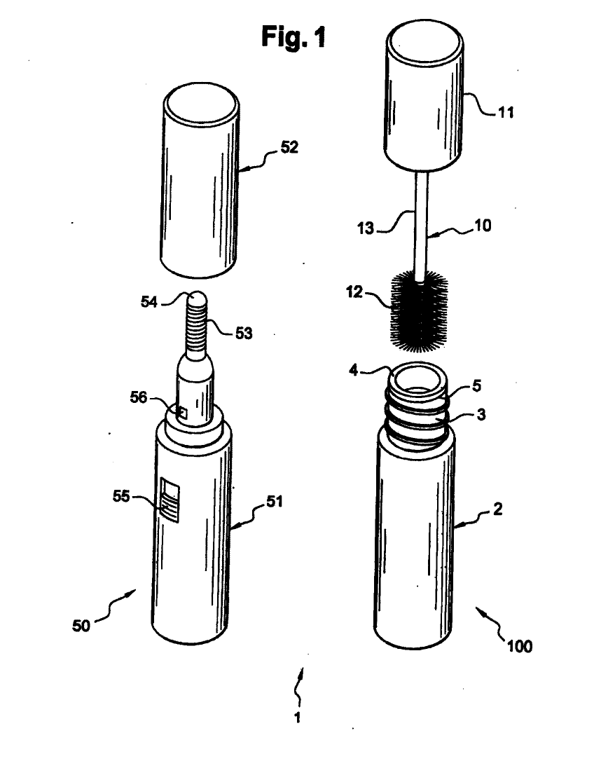

As depicted in FIG. 1, the composition according to the present disclosure may be packaged in a packaging and application assembly (1) comprising:
i) a container (2);
ii) a device (10) for applying the composition; and
iii) heating means (53) to raise the temperature of the composition to a temperature above its melting point, such as above or equal to its end melting temperature, simultaneously with or subsequent to its application.

For example, another subject of the present disclosure is a packaging and application assembly (1) for a makeup and/or care composition, such as for the eyelashes or the eyebrows, comprising:
i) a container (2);
ii) a makeup and/or care composition comprised inside the container, wherein the composition comprises at least one amorphous film-forming polymer capable of forming a water-soluble film,
iii) a device (10) for applying the makeup and/or care composition; and
iv) heating means (53) to raise the temperature of the composition to a temperature above its melting point, such as above or equal to its end melting temperature, simultaneously with or subsequent to its application.

For example, the composition may comprise at least one compound that gives the composition a thermal profile wherein the melting peak has a half-height width Lf less than or equal to 10° C.

According to one aspect of the present disclosure, the heating means are formed by a device that is separate from the application device or member, the assembly being configured in the form of a packaging and application device also comprising a container comprising a composition in accordance with the present disclosure. Such a device may be packaged in packaging of the blister-pack type. The heating means may be of the type described in U.S. Pat. No. 6,009,884 or 5,853,010. Other devices configured in the form of a heating brush, in the case of the eyelashes, may also be used. Such devices are described for example, in U.S. Pat. No. 6,220,252.

The kit 1 described in FIG. 1 comprises a mascara packaging and application assembly (100) and a heating device (50), separate from the packaging and application assembly. The two devices (100) and (50) may be sold together in the same packaging, of blister-pack type. The unit (100) containing the product may be sold separately.

The packaging and application assembly (100) comprises a container (2), comprising the composition according to the present disclosure, on which is mounted a threaded collar (3), one free edge of which delimits an opening (4). In the opening (4) is mounted a draining member (5). The assembly (100) also comprises an application device (10) comprising a stopper (11) solidly fastened to a stem (13), one end of which comprises an applicator (12), generally configured in the form of an arrangement of fibers held between the two branches of a twisted iron wire. An inner surface of the stopper (11) is threaded so as to engage with the threading of the neck (3). Thus, when the applicator (12) and the stem (13) are inside the container (2), the threading of the stopper (11) engages with the threading of the neck (3) such that the stopper sealably closes the opening (4) of the container. Such packaging and application assemblies are known.

The heating device (50) is in accordance with that described in U.S. Pat. No. 6,009,884. It comprises a grip portion (51) and a lid (52). A battery is placed inside the grip portion (51) and is connected to a heating wire (53) configured in the form of a coil arranged on a stem (54). A “switch” (55) allows the device to be switched on and off. An LED (56), when it changes color, indicates that the device is at the required temperature, and is thus ready for use.

The power supply of the heating part via the battery may be 12 V. The power dissipated may be about 1 watt. The heating wire (53) may be made of a nickel/chromium alloy.

According to an aspect of the present disclosure, the mascara may be applied without heating in a conventional manner to the eyelashes using a brush (12), and is then heated after application: the user engages the heating part (53) of the device (50) on the eyelashes so as to bring the deposit of product to the melting temperature of the composition.

Upon cooling, the composition returns to its semi-crystalline state, and can do so very quickly on account of the low width of the melting peak. The eyelashes are set, in a long-lasting manner, in their desired curled configuration.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific examples are reported as precisely as possible. Any numerical value, however, inherently contain certain errors necessarily resulting from the standard deviation found in their respective testing measurements. The following examples are intended to illustrate the present disclosure without limiting the scope as a result. The percentages are given on a weight basis.

The following examples are intended to illustrate the invention in a non-limiting manner.

### EXAMPLES 1 TO 3

The mascara compositions below according to the invention were prepared:

| | Example 1 | Example 2 | Example 3 |
|---|---|---|---|
| Polystearyl acrylate (Intelimer | 12 | 12 | 12 |
| IPA 13-1 from Landec) | | | |
| Oxyethylenated (30 EO) glyceryl | 2 | 2 | 2 |
| monostearate (Tagat S from | | | |
| Goldschmidt) | | | |
| Hydroxyethylcellulose | 1.86 | 1.86 | 1.86 |
| Sulphopolyester (Eastman | *** | 15 | *** |
| AQ 38 S from Eastman Chemical) | | | |
| Sulphopolyester (Eastman | *** | *** | 15 |
| AQ 48 Ultra from Eastman | | | |
| Chemical) | | | |
| Sulphopolyester (Eastman | 15 | *** | *** |
| AQ 55S Ultra from | | | |
| Eastman Chemical) | | | |
| Acrylamide/sodium | 1.3 | 1.3 | 1.3 |
| 2-acrylamidomethyl- | | | |
| propanesulphonate copolymer as an | | | |
| inverse emulsion at 40% in a mixture | | | |
| of polysorbate, isohexadecane | | | |
| and sorbitan oleate | | | |
| (Simulgel 600 from SEPPIC) | | | |
| Pigments (Black iron oxide) | 7 | 7 | 7 |
| Preserving agents | qs | qs | qs |
| Water | qs 100 | qs 100 | qs 100 |

After application of each of these compositions to the eyelashes followed by heating the film of composition for a few seconds using a heating brush, these mascaras were judged as having good behaviour and allowing a significant improvement in the curling of the eyelashes.

### Example 4

A mascara having the composition below was prepared:

| Polystearyl acrylate (Intelimer IPA 13-1 from Landec) | 12 | g |
|---|---|---|
| Sulphopolyester (Eastman AQ 55S from) | 15 | g |
| Beeswax | 3 | g |
| Hydroxyethylcellulose | 1.6 | g |
| Acrylamide/sodium 2-acrylamidomethylpropanesulphonate | 0.95 | g |
| copolymer as an inverse emulsion at 40% in a mixture of | | |
| polysorbate, isohexadecane and sorbitan oleate (Simulgel | | |
| 600 from SEPPIC) | | |
| Simethicone | 0.1 | g |
| Oxyethylenated (30 EO) glyceryl monostearate | 2 | g |
| (Tagat S from Goldschmidt) | | |
| Glyceryl mono- and distearate (Tegin M from Goldschmidt) | 1 | g |
| Pigments (black iron oxide) | 7 | g |
| Ethanol | 3 | g |
| Preserving agents | qs | |
| water qs | 100 | g |

### Procedure

The aqueous phase was prepared by dissolving the preserving agents and then the water-soluble surfactant with heating. The pigments and the water-soluble polymers (hydroxyethylcellulose and Simulgel) were introduced and then dispersed for a few minutes. The emulsion was then formed by dispersing the heated aqueous phase in the molten fatty phase at about 85° C. The mixture was then gradually cooled to room temperature.

This mascara composition had a thermal profile characterized by the following parameters:

starting melting temperature To=34.3° C.

end melting temperature Tf=48° C.

half-height peak width Lf=4.5° C.

The melting point of the composition was 46° C.

### Example 5

A mascara having the composition below was prepared:

| | |
|---|---|
| Polyolefin wax (Performa V260 from New Phase Technologies) | 0.1% |
| Polystearyl acrylate (Intelimer IPA 13-1 from Landec | 5% |
| 2-Amino-2-methyl-1,3-propanediol | 0.5% |
| Stearic acid | 5.8% |
| Carnauba wax | 7.3% |
| D-panthenol | 0.5% |
| Black iron oxide | 7.1% |
| Hydroxyethylcellulose quaternized with 2,3- | 0.1% |
| epoxypropyltrimethylammonium chloride | |
| Hydroxyethylcellulose | 0.9% |
| Simethicone | 0.15% |
| Non-stabilized sodium polymethacrylate at 25% in water | 1% |
| (Darvan 7 from Vanderbilt) | |
| Gum arabic | 3.4% |
| Beeswax | 8.7% |
| Candelilla wax | 2.5% |
| Triethanolamine | 2.4% |
| Oxyethylene and oxypropylene polydimethyl/methylsiloxane | 0.2% |
| (Q2-S220 from Dow Corning) | |
| Hydrogenated cottonseed oil | 0.5% |
| Hydrogenated jojoba wax (from Desert Whale) | 0.5% |
| Ethyl acrylate/ethyl methacrylate crosslinked copolymer as a | 1% |
| protected aqueous dispersion (Daitosol 5000 AD from Daito | |
| Kasei) | |
| Preserving agents | 0.45% |
| Water qs | 100% |

What is claimed is:

1. A cosmetic composition comprising, in a physiologically acceptable medium, at least one first compound which gives the cosmetic composition a thermal profile wherein the melting peak has a mid-height width Lf less than or equal to 10° C., and at least one amorphous film-forming polymer capable of forming a water-soluble film, wherein the at least one amorphous film-forming polymer is present in an amount greater than or equal to the amount of the first compound.

2. The composition according to claim 1, wherein the melting peak has a starting melting temperature To of greater than or equal to 10° C.

3. The composition according to claim 2, wherein the starting melting temperature To is greater than or equal to 15° C.

4. The composition according to claim 3, wherein the starting melting temperature To is greater than or equal to 20° C.

5. The composition according to claim 1, wherein the melting peak has an end melting temperature Tf of less than or equal to 90° C.

6. The composition according to claim 5, wherein the end melting temperature Tf is less than or equal to 80° C.

7. The composition according to claim 6, wherein the end melting temperature Tf is less than or equal to 70° C.

8. The composition according to claim 1, wherein the melting peak has a melting point m.p. ranging from 20° C. to 80° C.

9. The composition according to claim 1, wherein the melting peak has a temperature amplitude, ΔT=Tf−To, of less than or equal to 30° C.

10. The composition according to claim 1, wherein the at least one first compound has a thermal profile wherein the melting peak has a mid-height width Lf less than or equal to 10° C.

11. The composition according to claim 10, wherein the at least one first compound is chosen from waxes, semi-crystalline polymers and oils thickened with a structuring agent.

12. The composition according to claim 1, wherein the at least one first compound is present in an amount ranging from 1% to 60% by weight, relative to the total weight of the composition.

13. The composition according to claim 12, wherein the at least one first compound is present in an amount ranging from 3% to 55% by weight, relative to the total weight of the composition.

14. The composition according to claim 13, wherein the at least one first compound is present in an amount ranging from 5% to 50% by weight, relative to the total weight of the composition.

15. The composition according to claim 14, wherein the at least one first compound is present in an amount ranging from 10% to 40% by weight, relative to the total weight of the composition.

16. The composition according to claim 11, wherein the at least one wax is chosen from olive wax obtained by hydrogenation of olive oil esterified with stearyl alcohol, stearyl alcohol, stearyl stearate, stearyl benzoate, bis(trimethylolpropane)tetrastearate, polyethoxylated fatty acids of Montan wax, bis(trimethylolpropane)tetrabehenate and dioctadecyl carbonate wax.

17. The composition according to claim 11, wherein the at least one first compound is an oil thickened with a structuring agent.

18. The composition according to claim 1, wherein the at least one amorphous film-forming polymer is chosen from polyesters obtained by polycondensation of at least one dicarboxylic acid with at least one polyol.

19. The composition according to claim 18, wherein the at least one dicarboxylic acid is chosen from aromatic dicarboxylic acids comprising a —SO₃M group wherein M is a metal ion.

20. The composition according to claim 19, wherein M is chosen from Na⁺, Li⁺ and K⁺ ions.

21. The composition according to claim 19, wherein the at least one aromatic dicarboxylic acid is chosen from sulphoisophthalic acid, sulphoterephthalic acid, sulphophthalic acid and 4-sulphonaphthalene-2,7-dicarboxylic acid.

22. The composition according to claim 18, wherein the at least one polyol is a diol.

23. The composition according to claim 22, wherein that the at least one diol is chosen from ethylene glycol, diethylene glycol, triethylene glycol, 1,3-propanediol, cyclohexanedimethanol and 1,4-butanediol.

24. The composition according to claim 1, wherein the at least one amorphous film-forming polymer has a glass transition temperature (Tg) ranging from 25° C. to 120° C.

25. The composition according to claim 24, the at least one amorphous film-forming polymer has a glass transition temperature (Tg) greater than or equal to 38° C.

26. The composition according to claim 1, wherein the at least one amorphous film-forming polymer is present in an amount ranging from 0.1% to 40% by weight, relative to the total weight of the composition.

27. The composition according to claim 26, wherein the at least one amorphous film-forming polymer is present in an amount ranging from 5% to 30% by weight, relative to the total weight of the composition.

28. The composition according to claim 27, wherein the at least one amorphous film-forming polymer is present in an amount ranging from 10% to 20% by weight, relative to the total weight of the composition.

29. The composition according to claim 1, further comprising an aqueous phase.

30. The composition according to claim 29, wherein the aqueous phase is present in an amount ranging from 5% to 95% by weight, relative to the total weight of the composition.

31. The composition according to claim 29, wherein the aqueous phase is thickened with at least one thickener.

32. The composition according to claim 1, further comprising at least one additional film-forming polymer.

33. The composition according to claim 32, wherein the at least one additional film-forming polymer is present in a solids content amount ranging from 0.1% to 60% by weight, relative to the total weight of the composition.

34. The composition according to claim 33, wherein the at least one additional film-forming polymer is present in a solids content amount ranging from 0.5% to 40% by weight, relative to the total weight of the composition.

35. The composition according to claim 34, wherein the at least one additional film-forming polymer is present in a solids content amount ranging from 1% to 30% by weight, relative to the total weight of the composition.

36. The composition according to claim 1, further comprising at least one dyestuff.

37. The composition according to claim 36, wherein the at least one dyestuff is present in an amount ranging from 0.1% to 20% by weight, relative to the total weight of the composition.

38. The composition according to claim 37, wherein the at least one dyestuff is present in an amount ranging from 1% to 15% by weight, relative to the total weight of the composition.

39. The composition according to claim 1, wherein the composition is in a form for coating keratin fibers.

40. A cosmetic composition comprising, in a physiologically acceptable medium, at least one semi-crystalline polymer which gives the composition a thermal profile wherein the melting peak has a mid-height width Lf less than or equal to 20° C., and at least one amorphous film-forming polymer capable of forming a water-soluble film.

41. The composition according to claim 40, wherein the at least one semi-crystalline polymer is present in an amount ranging from 1% to 60% by weight, relative to the total weight of the composition.

42. The composition according to claim 41, wherein the at least one semi-crystalline polymer is present in an amount ranging from 3% to 55%, by weight, relative to the total weight of the composition.

43. The composition according to claim 42, wherein the at least one semi-crystalline polymer is present in an amount ranging from 5% to 50% by weight, relative to the total weight of the composition.

44. The composition according to claim 43, wherein the at least one semi-crystalline polymer is present in an amount ranging from 10% to 40% by weight, relative to the total weight of the composition.

45. The composition according to claim 40, wherein the at least one semi-crystalline polymer is chosen from copolymers resulting from the polymerization of at least one monomer comprising a crystallizable chain chosen from saturated C₁₄ to C₂₄ alkyl (meth)acrylates, C₁₁ to C₁₅ perfluoroalkyl (meth)acrylates, C₁₄ to C₂₄ N-alkyl(meth)acrylamides optionally with fluorine, vinyl esters comprising C₁₄ to C₂₄ alkyl or perfluoroalkyl chains, vinyl ethers comprising C₁₄ to C₂₄ alkyl or perfluoroalkyl chains, C₁₄ to C₂₄ alpha-olefins, para-alkylstyrenes with an alkyl group comprising from 12 to 24 carbon atoms, with at least one optionally fluorinated C₁ to C₁₀ monocarboxylic acid ester or amide of formula (II): 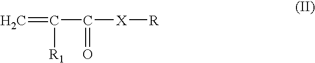
wherein R₁ is chosen from hydrogen atoms and CH₃ groups, R is chosen from optionally fluorinated C₁-C₁₀ alkyl groups and X is chosen from oxygen atoms, and NH and NR₂ groups wherein R₂ is chosen from optionally fluorinated C₁-C₁₀ alkyl groups.

46. The composition according to claim 40, wherein the at least one amorphous film-forming polymer is chosen from polyesters obtained by polycondensation of at least one dicarboxylic acid with at least one polyol.

47. The composition according to claim 46, wherein the at least one dicarboxylic acid is chosen from aromatic dicarboxylic acids comprising a —SO₃M group wherein M is a metal ion.

48. The composition according to claim 47, wherein M is chosen from Na⁺, Li⁺ and K⁺ ions.

49. The composition according to claim 46, wherein the at least one aromatic dicarboxylic acid is chosen from sulphoisophthalic acid, sulphoterephthalic acid, sulphophthalic acid and 4-sulphonaphthalene-2,7-dicarboxylic acid.

50. The composition according to claim 46, wherein that the at least one polyol is a diol.

51. The composition according to claim 50, wherein that the at least one diol is chosen from ethylene glycol, diethylene glycol, triethylene glycol, 1,3-propanediol, cyclohexanedimethanol and 1,4-butanediol.

52. The composition according to claim 40, wherein the at least one amorphous film-forming polymer has a glass transition temperature (Tg) ranging from 25° C. to 120° C.

53. The composition according to claim 52, the at least one amorphous film-forming polymer has a glass transition temperature (Tg) greater than or equal to 38° C.

54. The composition according to claim 40, wherein the at least one amorphous film-forming polymer is present in an amount ranging from 0.1% to 40% by weight, relative to the total weight of the composition.

55. The composition according to claim 54, wherein the at least one amorphous film-forming polymer is present in an amount ranging from 5% to 30% by weight, relative to the total weight of the composition.

56. The composition according to claim 55, wherein the at least one amorphous film-forming polymer is present in an amount ranging from 10% to 20% by weight, relative to the total weight of the composition.

57. The composition according to claim 40, wherein the at least one semi-crystalline polymer and the at least one amorphous film-forming polymer are present in a weight ratio of amorphous film-forming polymer to semi-crystalline polymer ranging from 0.3 to 3.

58. The composition according to claim 57, wherein the weight ratio of amorphous film-forming polymer to semi-crystalline polymer ranges from 0.6 to 2.

59. The composition according to claim 58, wherein the weight ratio of amorphous film-forming polymer to semi-crystalline polymer ranges from 0.9 to 1.5.

60. The composition according to claim 40, further comprising an aqueous phase.

61. The composition according to claim 60, wherein the aqueous phase is present in an amount ranging from 5% to 95% by weight, relative to the total weight of the composition.

62. The composition according to claim 60, wherein the aqueous phase is thickened with at least one thickener.

63. The composition according to claim 40, further comprising at least one additional film-forming polymer.

64. The composition according to claim 63, wherein the at least one additional film-forming polymer is present in a solids content amount ranging from 0.1% to 60% by weight, relative to the total weight of the composition.

65. The composition according to claim 64, wherein the at least one additional film-forming polymer is present in a solids content amount ranging from 0.5% to 40% by weight, relative to the total weight of the composition.

66. The composition according to claim 65, wherein the at least one additional film-forming polymer is present in a solids content amount ranging from 1% to 30% by weight, relative to the total weight of the composition.

67. The composition according to claim 40, further comprising at least one dyestuff.

68. The composition according to claim 67, wherein the at least one dyestuff is present in an amount ranging from 0.1% to 20% by weight, relative to the total weight of the composition.

69. The composition according to claim 68, wherein the at least one dyestuff is present in an amount ranging from 1% to 15% by weight, relative to the total weight of the composition.

70. The composition according to claim 40, wherein the composition is in a form for coating keratin fibers.

71. A non-therapeutic cosmetic process for making up or caring for keratin materials, comprising the application to the keratin materials of a cosmetic composition comprising, in a physiologically acceptable medium, at least one first compound which gives the cosmetic composition a thermal profile wherein the melting peak has a mid-height width Lf less than or equal to 10° C., and at least one amorphous film-forming polymer capable of forming a water-soluble film, wherein the at least one amorphous film-forming polymer is present in an amount greater than or equal to the amount of the first compound, and further wherein the cosmetic composition is brought to a temperature above or equal to its melting point, prior to, simultaneously with or subsequent to its application.

72. The process according to claim 71, wherein the cosmetic composition is brought to a temperature above or equal to its end melting temperature prior to, simultaneously with or subsequent to its application to the keratin materials.

73. A non-therapeutic cosmetic process for making up or caring for keratin materials, comprising the application to the keratin materials of a cosmetic composition comprising at least one amorphous film-forming polymer, wherein the cosmetic composition is brought to a temperature above or equal to its melting point, simultaneously with or subsequent to its application, to the keratin materials.

74. The process according to claim 73, wherein the cosmetic composition is brought to a temperature above or equal to its end melting temperature, simultaneously with or subsequent to its application to the keratin materials.

75. The process according to claim 71, wherein the cosmetic composition is brought to a temperature above or equal to its melting point, simultaneously with or subsequent to its application to the keratin materials, by an application device comprising heating means.

76. The process according to claim 73, wherein the cosmetic composition is brought to a temperature above or equal to its melting point, simultaneously with or subsequent to its application to the keratin materials, by an application device comprising heating means.

77. A non-therapeutic cosmetic process for making up or caring for keratin materials, comprising the application to the keratin materials of a cosmetic composition comprising, in a physiologically acceptable medium, at least one first compound which gives the cosmetic composition a thermal profile wherein the melting peak has a mid-height width Lf less than or equal to 20° C., and at least one amorphous film-forming polymer capable of forming a water-soluble film, wherein the said composition is brought to a temperature above or equal to its melting point m.p., simultaneously with or subsequent to its application to the keratin materials.

78. The process according to claim 77, wherein the cosmetic composition is brought to a temperature above or equal to its end melting temperature Tf, simultaneously with or subsequent to its application to the keratin materials.

79. A packaging and application assembly for a makeup and/or care composition for keratin materials, comprising:
i) a container;
ii) a makeup and/or care composition comprised inside the container, wherein the composition inside the container comprises at least one amorphous film-forming polymer capable of forming a water-soluble film,
iii) a device for applying the makeup and/or care composition; and
iv) heating means to raise the temperature of the composition to a temperature above its melting point, simultaneously with or subsequent to its application.

80. The packaging and application assembly of claim 79, wherein the keratin materials are chosen from eyelashes and eyebrows.

81. The packaging and application assembly of claim 79, wherein the heating means raises the temperature of the composition above or equal to its end melting temperature, simultaneously with or subsequent to its application.

82. A process for coating keratin fibers in order to deposit a film on the keratin fibers, wherein the film is uniform and/or has improved curling properties, said process comprising applying to fibers a cosmetic composition comprising, in a physiologically acceptable medium, at least one first compound which gives the cosmetic composition a thermal profile wherein the melting peak has a mid-height width Lf less than or equal to 10° C., and at least one amorphous film-forming polymer capable of forming a water-soluble film, wherein the amorphous film-forming polymer is present in an amount greater than or equal to the amount of the first compound.

83. A process for coating keratin fibers in order to deposit a film on the keratin fibers, wherein the film is uniform and/or has improved curling properties, said process comprising applying to fibers a cosmetic composition comprising, in a physiologically acceptable medium, at least one semi-crystalline polymer which gives the cosmetic composition a thermal profile wherein the melting peak has a mid-height width Lf less than or equal to 20° C., and at least one amorphous film-forming polymer capable of forming a water-soluble film.
